(19) United States
(12) Patent Application Publication
(10) Pub. No.: US 2012/0150160 A1
(43) Pub. Date: Jun. 14, 2012

(54) Apparatus for laser surgical ophthalmology

(76) Inventors: Klaus Vogler, Eschenau (DE); Christof Donitzky, Eckental/Eschenau (DE)

(21) Appl. No.: 13/365,449

(22) Filed: Feb. 3, 2012

(30) Foreign Application Priority Data
Aug. 3, 2009 (EP) PCT/EP2009/005605

(51) Int. Cl. A61F 9/011; A61F 9/01

(52) U.S. Cl. 606/4

(57) ABSTRACT

The invention relates to an apparatus for laser surgical ophthalmology, comprising a source of pulsed femtosecond laser radiation, components for guiding and focusing the laser radiation onto or into a tissue of an eye to be treated, a control unit controlling the source, which has been set up to switch the source between at least two operating modes with respectively differing radiation properties of the laser radiation, wherein in a first operating mode the radiation properties of the laser radiation are matched to the placement of an incision in the tissue, and in a second operating mode the radiation properties of the laser radiation are matched to a welding or cross-linking of the tissue.

This application claims priority to internationally filed application PCT/EP2009/005605 filed on Aug. 3, 2009.

The invention relates to an apparatus for laser surgical ophthalmology.

In refractive ophthalmological surgery the refractive properties of the eye are changed by interventions on the eye of a patient for the purpose of correcting sight defects. Known, in particular, is the so-called LASIK procedure (laser in-situ keratomileusis), wherein the surface curvature of the patient's cornea is changed. In this procedure, in a first operative step a flat corneal incision is made. In the process a small cover disc, designated as a flap, arises, which is folded upwards so that the underlying corneal tissue (stroma) is exposed. In the ensuing part of the LASIK operation, material is abraded from the stroma with a laser in accordance with a defined ablation pattern. After this, the flap is folded back and covered again relatively quickly with the corneal epithelium.

For the purpose of generating the flap incision, for some time a so-called femtosecond laser has been employed which makes the flat incision, described above, in the cornea. To this end, the laser is focused within a plane below the surface of the cornea and is guided on a path within this plane in order to generate the flap. The extremely short laser pulses within the femtosecond range which are used in this procedure have such high powers that, given suitable focusing, an incision can be made by utilising the so-called photodisruptive effect.

These femtosecond laser-instrument systems are also employed for keratoplasties. In this operation on the cornea, diseased corneal tissue, for example, is replaced by suitable donor material (transplantation), in order to reduce cases of ametropia. The incisions that are necessary in this connection on the tissue of the donor cornea and of the recipient cornea can be obtained in fundamentally the same manner by a guidance of precisely focused femtosecond pulses, utilising the photodisruptive effect, as is also the case with the femtosecond flap incisions of LASIK.

The segment of corneal tissue transferred from the donor is fixed manually by means of needle and monofilament nylon thread.

In addition, femtosecond laser-instrument systems are also being increasingly employed for difficult femtosecond laser keratoplasties and corneal-ring-segment incisions (intrastromal corneal ring segments, also called INTACS), since a number of advantages arise in comparison with the conventional surgical interventions of this type. A more accurate guidance of the incision, independent of the individual ability of the operating surgeon, and an accurately matching identity between donor corneal segment and acceptor corneal segment are available. Arbitrary incision shapes for the grafts and, in particular, suitably fashioned marginal incisions and marking incisions, which facilitate an anchorage of the donor-cornea segment in the acceptor segment of the cornea, can also be realised, such as, for instance, top-hat, mushroom, Christmas-tree or zigzag geometries. These marginal-incision geometries improve the anchorage of the donor cornea in the cornea of the recipient and offer a good sealing of the incision region. Furthermore, partially lamellar and posterior corneal grafts can also be carried out distinctly more easily by means of femtosecond-laser keratoplasty.

Overall, however, at the current time a number of disadvantages are still associated with the present grafting procedure. Despite improved and automated incision techniques and geometries, the manually-placed suture often does not result in a completely tight closure of the wound; furthermore, it frequently induces an undesirable astigmatism or other higher-order sight defects. Besides, as a manual procedure the suturing of the corneal segments is relatively time-consuming and requires a more lengthy aftercare, and the outcome is still dependent on the manual skill of the operating surgeon.

Alternative procedures—such as an adhesion by means of chemical additive substances such as, for instance, cyanoacrylate—also have fundamental disadvantages, such as, for instance, low durability, poor compatibility and also the introduction of foreign substances which are alien to the body. Besides, adhesion also requires concentrated manual labour on the part of the operating surgeon.

It is the object of the invention to make an apparatus available that enables an extensive automation of the individual working steps in the course of a keratoplasty.

With a view to achieving this object, in accordance with the invention an apparatus for laser surgical ophthalmology is provided, comprising: a source of pulsed femtosecond laser radiation, components for guiding and focusing the laser radiation onto or into a tissue of an eye to be treated, a control unit controlling the source, which has been set up to switch the source between at least two operating modes with respectively differing radiation properties of the laser radiation, wherein in a first operating mode the radiation properties of the laser radiation are matched to the placement of an incision in the tissue and in a second operating mode the radiation properties of the laser radiation are matched to a welding of tissue. The pulsed femtosecond laser radiation generated by the source is directed via the components for guiding and focusing the laser radiation onto, for example, the cornea of the eye to be treated. If the apparatus is in the first operating mode, the radiation properties of the femtosecond laser radiation are matched in such a way that upon impinging on the tissue it generates, for example, the photodisruptive effect and in this way, given appropriate beam guidance, separates the tissue and gives rise to an incision. In this operating mode the flap incision mentioned in the introduction, for example, can be generated. In this connection the settings of the femtosecond laser radiation, which, for example, are slightly different for a flap-bed incision and for the flap-margin incision as regards the spacing or the temporal succession of individual focal points of the laser, are intended to be encompassed by the first operating mode.

In the second operating mode, on the other hand, the radiation properties of the femtosecond laser radiation have been modified to such an extent in comparison with the first operating mode that the laser radiation brings about on or in the tissue a welding or an adhesion or cross-linking of individual tissue parts, brought about by reorientation of the cell fibrils. This can, for example, be brought about by the tissue being brought into a temperature range that lies below a denaturation threshold or coagulation threshold of the tissue. In the case of corneal tissue, this temperature range lies, for example, between 55° C. and 65° C. In the case of corneal tissue in this connection, the extracellular, intralamellar structure of the cornea is modified in such a manner that in a subsequent cooling process a reorganisation of the location of the collagen fibrils and of the structural cross-linking thereof occurs. The collagen fibrils do not denature in this process but appear to cross-link anew by virtue of the rise in temperature and the subsequent cooling.

Alternatively or additionally to the welding or new cross-linking by means of the temperature cycle that has been described, a welding can also be effected by cross-linking by means of UV radiation. As a result of absorption of UV radiation, for example with a wavelength of 380 nm, a local photo-induced formation of radicals and an increase in temperature may occur, as a result of which, for example, the collagen fibrils of the cornea can likewise be restructured and a reinforcement or reforming of their cross-linking occurs. The absorption of the UV radiation may take place, for example, on riboflavin which is present in the tissue.

For a successful welding or cross-linking of tissue, low-energy pulses with high repetition frequency below the LIOB threshold (LIOB=laser-induced optical breakdown) and with pulse durations within the femtosecond range may be employed, which generate a direct or indirect thermal action in transparent materials and tissues.

Possible fields of application of femtosecond welding are—in addition to application in the grafting of corneal tissue, that is to say, the welding of the donor-tissue segment with the acceptor corneal bed—also the reattachment of the flap in the case of a femtosecond LASIK incision in the marginal region after the refraction-correcting structuring of the stroma. The flap that has been folded back may be welded to the residual stroma again over the entire marginal region or at individual marginal points. Furthermore, it is also possible to carry out cross-linking operations or local welding operations on lower-lying corneal layers or other tissue structures of the eye, without a direct opening (trepanation) being necessary.

The radiation properties of the laser radiation provided for the second operating mode of the apparatus may differ from those of the first operating mode as regards various parameters.

A first such beam parameter is the fluence per radiation pulse, which may be lower in the second operating mode than in the first operating mode. The fluence, which is defined as energy per unit area and represents a measure of the energy given off to the tissue, may, in particular, lie distinctly below the fluence threshold at which the photodisruptive effect occurs. In the case of a femtosecond LASIK incision or a keratoplasty incision on the human cornea this threshold amounts to 0.3 Joule/cm² to 1 Joule/cm².

Furthermore, there is provision to employ a lower pulse energy in the second operating mode than in the first operating mode. The energy per pulse that, for example, is employed in the course of an incision, that is to say in the first operating mode, amounts to approximately 0.2 μJ to 1.5 μJ. For the use of the femtosecond laser pulses for the welding or adhesion of tissue, on the other hand, pulse energies within the range of 10-500 nJ are provided. Crucial in this connection is the fluence of the laser radiation, which in the second operating mode has to lie below the LIOB threshold. In the case of a larger focus, the energy may also be higher. A relatively large irradiation diameter may be an advantage for a secure overlap of the welding points.

Whereas in the first operating mode, which is designed for the placing of an incision, a clear separation of the individual focal points of the laser is preferably provided in the case of a focus diameter of about 2-5 μm and an intermediate spacing of the individual focus radii of 1-2 μm (corresponding to a focus centre-to-centre spacing of 2-10 μm), the strategy in the second operating mode provides that several pulses act on the same point in the tissue, for example the cornea. This brings about a thermal accumulation of the pulse energies. In this connection there may be provision that between 2 and 10,000 pulses impinge on virtually the same point. The exact number of pulses depends on the size of the laser focus, on the individual energy of the pulse and also on further factors such as, for instance, the transparency or absorption of the tissue.

There may, in particular, be provision that the laser radiation possesses a larger focus size in the second operating mode than in the first operating mode. In particular, focus-location diameters between 5 μm and a maximum of 50 μm are provided. Where appropriate, blurred focus regions may also be employed, that is to say, the focus diameter is larger than the incision interspace between donor margin and acceptor margin, in order to enable a welding of the two tissue parts to one another. This can be assisted by a spatial overlapping of the individual laser pulses, for example by a reduced speed of the scan deflection in comparison with the first operating mode.

In another embodiment there may be provision that the laser radiation possesses a different, in particular lower, pulse repetition rate in the first operating mode than in the second operating mode. Whereas in the first operating mode, which is designed for the placing of an incision, pulse repetition rates of, for example, 60-500 kHz, in particular up to 1 MHz, are provided, in the second operating mode the use of repetition rates from 200 kHz to 10 MHz has turned out to be advantageous.

According to one embodiment of the apparatus according to the invention there is provision that the components for guiding and focusing the laser radiation include a scanner unit, and the control unit has been set up for the purpose of controlling the scanner unit in such a manner that temporally consecutive pulses of the laser radiation in the second operating mode succeed one another locally more closely at the target region than in the first operating mode. This enables an overlapping of the individual laser pulses, for example by virtue of a reduced beam-deflection speed, and in this way a uniform overlap of the tissue region to be welded or cross-linked.

In this connection there may, in particular, be provision that the control unit has been set up for the purpose of controlling the scanner unit in such a manner that temporally consecutive pulses of the laser radiation in the second operating mode overlap one another completely or at least partially at the target region. This overlapping of the laser pulses results in a thermal accumulation of the individual pulse energies, and in this way enables a targeted, stepwise increase in the local tissue temperature until the desired temperature window has been attained. In this manner the thermal conductivity and the thermal capacity of the tissue to be treated locally can be taken into consideration, and in this way a targeted drive of the tissue temperature can be obtained.

In this context it is, in particular, an advantage that the apparatus exhibits sensorics, connected to the control unit, for registering the temperature of the irradiated tissue, the control unit having been set up to control, at least in the second operating mode, the radiation properties and/or the motion of the laser radiation over the tissue to be treated, in a manner depending on the registered temperature. In the case of the sensorics for registering the temperature, it may, for example, be a question of a radiation sensor registering the tissue temperature in non-contacting manner. Alternatively, it may also be a question of an indirect registration of the temperature via the energy introduced into the tissue, or the temperature may be ascertained, as far as possible, by a direct contact of a temperature sensor with the tissue to be treated or with the environment thereof. By means of these sensorics it is possible to obtain as accurate as possible a temperature profile of the tissue for the purpose of generating the desired new cross-linking. In this connection, in particular a precise drive of the desired temperature window can be obtained by a feedback of the attained temperature in connection with the control of the pulse parameters.

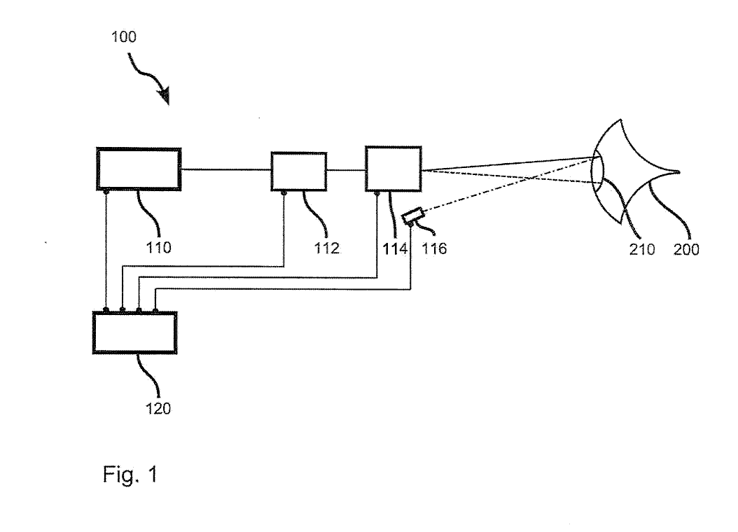

The invention will be elucidated further in the following on the basis of the appended drawing. The single FIG. 1 represents an embodiment of a laser apparatus according to the invention.

The apparatus 100 represented in FIG. 1 for laser surgical ophthalmology includes by way of laser radiation source a working laser 110 that is suitable, for example, for corneal treatments and that emits pulsed laser radiation within the femtosecond range. Components for beam guidance and for focusing the laser radiation are schematically combined in component 112. Furthermore, a scanner device 114 is provided which guides the laser beam along a desired scan profile in or on the cornea 210 of the schematically represented eye 200. For the purpose of registering the temperature profile generated by the pulsed laser radiation on or in the cornea 210, a temperature sensor 116 is provided. The working laser 110, the beam-guiding-and-shaping components 112, the scanner device 114 and the temperature sensor 116 are connected to a control unit 120 via suitable control lines. The control unit 120 has been set up to vary the beam parameters of the pulsed laser radiation via a suitable drive of the working laser 110 and of the beam-guiding-and-focusing components 112 in such a way that in a first operating mode the radiation properties are matched to the placing of an incision, for example in the tissue of the cornea 210. In a second operating mode, on the other hand, the beam parameters can be matched in such a way that the implementation of a welding or cross-linking of tissue is possible.

In order, for example, to carry out a penetrating keratoplasty, the following procedure is accordingly possible. Using the first operating mode, which encompasses defined properties of the beam-guiding, imaging and focusing optics 112, the apparatus 100 could firstly perform the resection of the corneal segment of the cornea 210 in question, using a suitable drive of the working laser 110. The beam parameters selected by the first operating mode would enable a photodisruptive application of an incision. This resection is to be carried out both on the donor cornea and on the recipient cornea or acceptor cornea. In this connection it is, in particular, conceivable to record the incision geometry provided for the incision in the donor cornea and to employ these data in the preparation of the corneal bed in the recipient cornea. Alternatively, the reverse sequence may also have been provided, that is to say, firstly preparation of the recipient cornea and subsequently resection of the donor cornea.

After the corneal segment of the donor has been taken away, said segment can be inserted into the corneal bed of the acceptor. In this connection the incision geometries mentioned in the introduction may be advantageous in connection with the orientation and mechanical linkage. For the subsequent fixing of the implant, the apparatus 100 is set to the second operating mode. The laser parameters that are now available bring about a cross-linking of the tissue segments situated above one another and weld them together. Consequently the manual procedural portion of the suturing or adhesion of the cornea onto the donor part with the remaining cornea of the recipient is replaced by a largely automatable configuration of the procedure. Consequently the removal of the diseased part of the cornea and/or the adequate incision in the donor part by virtue of femtosecond laser incisions and also the ultimate fixing of the corneal donor in the cornea of the acceptor by virtue of a corneal welding with the same femtosecond laser source 110 are performed with a distinctly reduced proportion of manual interventions by the operating surgeon.

This does not just bring about an increased automation of the operation, which as a rule guarantees better reproducibility of the results. Above and beyond this, the femtosecond working-laser source 110, together with the associated components 112 and the scan unit 114, is made available for a further application.

In the course of the implementation of the tissue welding, via the sensor 116 the increase in temperature in the cornea 210 that is brought about by the laser radiation of the working laser 110 can be recorded and communicated to the control instrument 120. Via the registered temperature values the control instrument can undertake a suitable adaptation, for example of the pulse rate, of the pulse overlap or of the pulse energy. In the present exemplary embodiment the temperature sensor 116 is represented outside the optical axis of the laser radiation of the working laser 110. But the temperature sensor 116 may alternatively also be integrated into the primary beam path.

In addition to the aforementioned example of a penetrating keratoplasty, partial lamellar and posterior corneal grafts as well as, for example, a fixing of the flap after the folding-back of the same in the course of the femtosecond LASIK operation can also be carried out. Furthermore, a more extensive application of the tissue welding to other ocular-tissue structures is also conceivable.

1. Apparatus (100) for laser surgical ophthalmology, comprising
a source (110) of pulsed femtosecond laser radiation,
components (120) for guiding and focusing the laser radiation onto or into a tissue (210) of an eye (200) to be treated,
a control unit (120) controlling the source (110), which has been set up to switch the source (110) between at least two operating modes with respectively differing radiation properties of the laser radiation,
wherein in a first operating mode the radiation properties of the laser radiation are matched to the placement of an incision in the tissue (210) and
in a second operating mode the radiation properties of the laser radiation are matched to a welding of tissue or cross-linking of tissue.

2. Apparatus according to claim 1, wherein the laser radiation possesses a higher fluence per pulse in the first operating mode than in the second operating mode.

3. Apparatus according to one of the preceding claims, wherein the laser radiation possesses a different, in particular higher, pulse energy in the first operating mode than in the second operating mode.

4. Apparatus according to one of the preceding claims, wherein the laser radiation possesses a different, in particular smaller, focus size in the first operating mode than in the second operating mode.

5. Apparatus according to one of the preceding claims, wherein the laser radiation possesses a different, in particular lower, pulse repetition rate in the first operating mode than in the second operating mode.

6. Apparatus according to one of the preceding claims, wherein the components for guiding and focusing the laser radiation include a scanner unit (114), and the control unit (120) has been set up for the purpose of controlling the scanner unit (114) in such a manner that temporally consecutive pulses of the laser radiation in the second operating mode succeed one another locally more closely at the target region than in the first operating mode.

7. Apparatus according to claim 6, wherein the control unit (120) has been set up for the purpose of controlling the scanner unit (114) in such a manner that temporally consecutive pulses of the laser radiation in the second operating mode overlap one another at least partially at the target region.

8. Apparatus according to one of the preceding claims, further with sensorics, connected to the control unit (120), for registering (116) the temperature of the irradiated corneal tissue, the control unit (120) having been set up to control, at least in the second operating mode, the radiation properties or/and the motion of the laser radiation over the tissue to be treated, in a manner depending on the registered temperature.
